(19) United States
(12) Patent Application Publication
(10) Pub. No.: US 2003/0125347 A1
(43) Pub. Date: Jul. 3, 2003

(54) Pharmaceutical composition

(75) Inventors: Kirsten A. Anderson, Dublin (IE); James M. Hoch, San Francisco, CA (US); Gary Liversidge, West Chester, PA (US); Jean Marie Duvall, Portola Valley, CA (US); Joshua R. Slavitt, Merion Station, PA (US)

Correspondence Address: SYNNESTVEDT & LECHNER, LLP, 2600 ARAMARK TOWER, 1101 MARKET STREET, PHILADELPHIA, PA, 191072950

(73) Assignee: Elan Corporation plc, Dublin (IE)

(21) Appl. No.: 10/288,262

(22) Filed: Nov. 4, 2002

Related U.S. Application Data
(60) Provisional application No. 60/340,111, filed on Nov. 2, 2001.

(51) Int. Cl. A61K 49/00; A61K 35/78; A61K 31/485

(52) U.S. Cl. 514/282; 424/10.1; 424/760

(57) ABSTRACT

A pharmaceutical composition and an oral dosage form are disclosed comprising an opiate and an irritant. A method is also described for discouraging abuse of an opiate comprising combining a therapeutically effective amount of an opiate and an irritant into an oral dosage form. A method is also described for treating pain by administering a therapeutically effective amount of an opiate and an irritant in an oral dosage form.

### FIELD OF THE INVENTION

Opiates have long been recognized as effective in the treatment of pain. Considered chemically as alkaloids, opiates were derived originally from the opium poppy (papaver somniferum). A wide variety of opiates have since been synthesized for their therapeutic value as analgesics.

In addition to their analgesic action, opiates can also cause other effects such as, for example, euphoria and respiratory depression. At high dosage levels, the respiratory depressive effect can be fatal. Opiates have also been found to cause physical dependence. Thus, while specific therapeutically effective dosages vary by particular opiate and context of use, it has been recognized that careful administration of opiates is critical in achieving effective pain relief while avoiding the deleterious health effects that can result from high dosage levels and physical dependence.

It is known that modified-release formulations of opiate-based analgesics have certain advantages over opiate-based analgesics that do not have such modified-release properties. Among these advantages are the need for less frequent administration and effective pain relief over an extended period of time. Because of the extended period of time over which the opiate is released from such formulations, the individual dosage forms often contain much larger amounts of opiates as compared to formulations which do not have modified-release properties. For example, oxycodone HCl is a commonly prescribed opiate that is dispensed in tablets which do not have modified-release properties and which contain 5 milligrams of oxycodone HCl. By contrast, when prescribed in a modified-release form, such tablets may contain from about 10 to about 80 milligrams or more of oxycodone HCl. It is, at least in part, by virtue of the modified-release properties of such formulations that the analgesic effect in a patient can be maintained effectively over an extended period of time.

With the synthetic enhancement and broader use of opiates as analgesics, opiates have become also more widely associated with addiction and abuse. The incidence of addiction and abuse of opiates has been noted in connection with a variety of opiate-containing oral dosage forms. Because of the relatively high levels of opiates contained within modified-release formulations, such formulations in particular have become the subject of abuse.

In order to achieve the euphoric effects associated with the abuse of opiate formulations, abusers will use amounts of opiates significantly greater than are commonly used in therapeutic applications, and will administer the opiates in a variety of known ways in order to effect a large and immediate release of opiates into their bodies. Immediate-release dosage forms may be abused by the consumption of a multiple number of tablets. For modified-release dosage forms which each generally contain a larger amount of opiate per unit dose, abusers will realize an immediate release of the opiate by consuming the oral dosage form and/or the contents thereof after destroying its modified-release properties, for example, by chewing the dosage form and swallowing the powder, by crushing the dosage form or its contents and ingesting the powder through nasal or oral inhalation or insufflation, or by ingesting or injecting a solution or suspension containing the opiate extracted from the dosage form. Through the destruction of the modified-release properties of modified-release dosage forms, an immediate release of the entire amount of opiate contained within the oral dosage form may be accomplished. The methods of opiate abuse described herein have been reported to be a significant social and health problem that has resulted in increases in addiction and deaths due to overdose.

### SUMMARY OF THE INVENTION

In accordance with one aspect of the present invention, there is provided a pharmaceutical composition comprising an opiate and an irritant. Opiates considered suitable for use in the composition of the present invention may be any opiate that is effective therapeutically for the treatment of pain. Irritants considered suitable for use in the composition of the present invention may be any substance which is acceptable for human consumption and which is capable of causing significant discomfort in a human either in the tissues of the body that come into contact with the irritant, systemically or both. The irritant may be provided either in a sub-clinical amount and/or in sequestered form. Preferably, the irritant is provided in a sub-clinical amount in compositions intended for use in immediate-release dosage forms, and in sequestered form in pharmaceutical compositions for use in modified-release dosage forms.

Another aspect of the present invention is an oral dosage form comprising an opiate and an irritant. The oral dosage form of the present invention may be provided such that the opiate contained therein is released immediately or such that the release of opiate from the dosage form is modified to permit the release of the opiate over an extended period of time. In either form, the irritant is provided such that it is substantially incapable of causing discomfort to a patient when the dosage form is administered as directed. If, however, the dosage form is used in a manner consistent with abuse, for example, by ingestion of either high doses of an immediate-release dosage form or a modified-release dosage form in which the modified-release properties have been destroyed, the irritant is capable of causing discomfort either locally, systemically or both sufficient to act as a deterrent to such use.

Another aspect of the present invention is a method for discouraging abuse of an opiate comprising combining a therapeutically effective amount of an opiate and an irritant into an oral dosage form. The method may employ the use of a sub-clinical amount of an irritant in an immediate-release dosage form or a sequestered irritant in a modified-release dosage form. Preferably, the dosage form employed in the method of the present invention will have a modified-release feature that is designed to permit the release of the opiate over an extended period of time.

A further aspect of the present invention is a method for treating pain by administering a therapeutically effective amount of an opiate and an irritant in an oral dosage form. In preferred form, he method employs a sub-clinical amount of an irritant in an immediate-release dosage form or a sequestered irritant in a modified-release dosage form. Preferably, the dosage form will have a modified-release feature that permits the release of the opiate over an extended period of time.

There are important advantages that stem from the composition, dosage form and methods of the present invention. When the dosage form is administered as directed, that is, at therapeutic doses or in a manner that maintains the structural integrity of the oral dosage form and/or the contents thereof, the irritant is substantially incapable of causing significant discomfort in the average individual. If, however, the dosage of immediate-release dosage forms is exceeded, or the structural integrity of a modified-release dosage form is destroyed and the opiate contained within is released, discomfort is caused sufficient to deter consumption in such a manner. As a result, the present invention allows for the beneficial therapeutic aspects of opiates to continue to be realized while, at the same time, reducing significantly the incidence of abuse associated with opiate-containing oral dosage forms.

### DETAILED DESCRIPTION OF THE INVENTION

The composition of the present invention comprises an opiate and an irritant.

The term “opiate” is defined for purposes of the present invention to include all opiates, opiate-based derivatives and compounds, and pharmaceutically acceptable salts thereof, suitable for use as a therapeutically effective analgesic, either alone or in combination with other substances. Examples of suitable opiates include alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, pro-poxyphene, sufentanil, tilidine, and tramadol.

Preferred opiates of the present invention are codeine, hydrocodone, hydromorphone, levorphanol, meperidine, morphine, and oxycodone, and pharmaceutically acceptable salts thereof. Particularly preferred opiates are hydrocodone, hydromorphone, and oxycodone and pharmaceutically acceptable salts thereof.

The opiate may include two or more opiate constituents. For example, two or more opiates having different properties, such as half-life, solubility, potency, and a combination of any of the foregoing may be used. Examples of preferred opiates in combination are hydrocodone and oxycodone and pharmaceutically acceptable salts thereof.

In preferred form, the composition comprises as the analgesic only one or more opiates. However, the composition may include also a non-opiate-based therapeutically active ingredient. Such a non-opiate-based therapeutically active ingredient may provide additional analgesia and include, for example: aspirin; acetaminophen; non-steroidal anti-inflammatory drugs (“NSAIDS”), for example, ibuprofen and ketoprofen; N-methyl-D-aspartate receptor antagonists, for example, a morphinan such as dextromethorphan or dextrorphan, or ketamine; cyclooxygenase-II inhibitors (“COX-II inhibitors”); glycine receptor antagonists; and prostaglandin synthesis inhibitors.

In yet other embodiments of the present invention, one or more non-opiate-based active ingredients may be included to provide an effect other than analgesia, for example, an antitussive, expectorant, decongestant, or antihistamine.

The opiate is included in the composition in a therapeutically effective amount. Such amount will vary in accordance with a number of factors including, for example, the particular species of opiate used, the presence of other ingredients, the specific form of the oral dosage formulation, and the particular application in which the composition is intended to be used. It is believed that in most applications, the amount of opiate included in the composition will be from about 0.1 to about 40 wt. %. In preferred form, the amount of opiate included in the composition will be from about 0.1 to about 30 wt. %, and even more preferably from about 0.1 to about 20 wt. %. When combined with other therapeutically active ingredients, the amount of opiate included in the composition will be from about 0.1 to about 40 wt. %, preferably from about 0.1 to about 30 wt. %, and even more preferably from about 0.1 to about 20 wt. %.

The term “irritant” is defined for purposes of the present invention as any substance which is acceptable for human consumption and which is capable of causing significant discomfort in a human, either locally and/or systemically. A substance is considered acceptable for human consumption if it is non-toxic at dosages which are capable of producing significant discomfort.

The term “significant discomfort” is defined for purposes of the present invention as mental or physical distress of sufficient magnitude as to be capable of influencing the opiate-consuming behavior of opiate abusers. The particular discomfort caused by the irritant may manifest its effects locally, that is, at the site of administration, and/or systemically. Examples of effects caused by local irritants include swelling, redness, burning or stinging of the buccal and/or nasal cavities, and localized neuromuscular pain at the injection site. Examples of effects caused by systemic irritants include gastric distress, allergic reaction, neuromuscular pain, cardiovascular distress, skin rash, respiratory distress, and psychological distress.

An important aspect of the irritant as provided in the oral dosage form of the invention is that it is substantially incapable of causing significant discomfort when it is consumed as part of an intact oral dosage form and administered within the prescribed dosage range. Administered in this manner, the presence of the irritant in the oral dosage form also does not substantially interfere with the proper therapeutic uses. When ingested in ways associated with abuse, however, the irritant is released in an amount sufficient to cause significant discomfort in the abuser.

In one embodiment, the irritant is provided in a sub-clinical amount either in a modified-release oral dosage form or, more preferably, in an immediate-release oral dosage form. The term “sub-clinical” is defined for purposes of the present invention as an amount of a substance which, if consumed, is insufficient to produce significant discomfort in the average individual. In such an embodiment, the analgesic effect of the dosage form is realized without causing significant discomfort when administered within the therapeutic dose range. If, however, the oral dosage form is ingested in an amount significantly greater than the therapeutic dose range, which is a mode of use associated with opiate abuse, the total amount of irritant introduced into the abuser's body is increased to a clinical amount, that is, to an amount sufficient to produce significant discomfort.

In another embodiment, the irritant is sequestered and provided in a modified-release oral dosage form. The term “sequestered” is defined for purposes of the present invention as physically isolated and/or chemically bound and biologically unavailable. In such an embodiment the modified-release properties of the dosage form are realized without causing significant discomfort. If, however, the modified-release properties of the dosage form are destroyed such as by physical destruction or dissolution, which is another mode of use associated with opiate abuse, then the irritant is released from sequestration and is capable of causing significant discomfort.

The irritant may be sequestered in a variety of ways all of which are considered within the scope of the invention. Physical sequestration may be achieved, for example, by coating the irritant in a pharmaceutically acceptable material that forms a substantially indigestible barrier. The coated irritant is then combined with the opiate to form a composition. Sequestration may be accomplished also by the formation of chemical bonds between the irritant and a pharmaceutically acceptable material, such as for example a chelating agent, such that the irritant is rendered biologically unavailable to the patient when taken as directed as a part of a dosage form. Whether physical and/or chemical sequestration is employed, the manner of sequestration is selected so that the irritant is released from sequestration if the physical barrier or the chemical bonds of the sequestering agent is compromised. The release of sequestered irritants may be accomplished physically, for example, by crushing, or chemically, for example, by a solvent capable of degrading the sequestering material or breaking the bonds with the irritant. By the selection of sequestering agents which are capable of releasing irritants by means of the same methods that are associated with abuse of pharmaceutical forms of opiates, the sequestration of irritants is specifically designed to deter such abuse.

Suitable irritants that cause local irritation may do so by causing pain in the tissues with which the irritants come into contact. If the oral dosage form includes a local irritant and is administered in powder form by nasal or oral inhalation or insufflation, or ingested as a powder, solution or suspension, the irritant may cause swelling, redness, itching, burning or stinging in the nasal and/or buccal tissues. If a solution or suspension of such an oral dosage form is injected, the irritant may cause localized dermal and/or neuromuscular pain in the area of the injection site.

Examples of suitable local irritants may be of natural or synthetic origin and include mustard and derivatives of mustard, for example, allyl isothiocyanate and p-hydroxybenzyl isothiocyanate; capsaicinoids such as capsaicin, dihydrocapsaicin, nordihydrocapsaicin, homocapsaicin, and homodihydrocapsaicin; mint; aspirin; and acids such as acids with one or more carboxyl moieties such as formic acid, acetic acid, propionic acid, butyric acid, valeric acid, caproic acid, caprilic acid, capric acid, oxalic acid, malonic acid, succinic acid, glutaric acid, adipic acid, maleic acid, fumaric acid, and citric acid. Preferred local irritants for use in the present invention are capsaicinoids such as, for example, capsaicin.

Suitable systemic irritants cause irritation by prompting discomfort in one or more physiological system without regard to the specific areas of the body which contact the irritant. Substances that are systemic irritants to the gastrointestinal system may be selected to cause excessive or insufficient salivation, nausea, emesis, cramping, gas pain or discomfort, dyspepsia, heartburn, and/or diarrhea. Examples of such irritants include emetics such as ipecac and chemotherapeutic agents, and laxatives such as aloe vera, bisacodyl, casanthranol, cascara sagrada, castor oil, dehydrocholic acid, phenolphthalein, senna and sennosides.

Substances that are systemic irritants to the neurological system may be selected to cause one or more effects such as headache, vertigo, and sensory discomforts such as foul odors and/or tastes. Examples of such irritants include sulfurous compounds and sulfur-containing materials, carboxylic acids having up to 10 carbon atoms, and other active compounds known to cause neurological discomfort as a side effect.

Substances that are systemic irritants to the pulmonary, dermatological and immune systems may be selected to cause one or more effects such as wheezing, shortness of breath, difficulty in breathing, coughing, sneezing, rhinorrhea, hives, skin rash, swelling or redness, and discomfort associated with redness, itching, swelling, or watering of eyes or nasal membranes. Examples of such irritants include histamines and other active compounds known to cause such discomforts as side effects.

Substances that are systemic musculoskeletal irritants may be selected to cause one or more effects such as muscle soreness, cramping, and joint pain. Examples of such irritants include diuretics, nifedipine, B₂ agonists such as terbutaline or albuterol, and other active compounds known to cause musculoskeletal discomfort as a side effect.

Substances that are suitable psychological irritants may be selected to cause one or more psychological effects such as paranoia or anxiety as well as associated physical symptoms such as rapid heartbeat, irregular breathing, dizziness, nervousness, and tremors. Examples of such irritants include aminophylline, heterocyclic antidepressants, antidyskinetics, anticholinergics such as atropine, beta-Z adrenergic agents such as isoproterenol and metaproterenol, cycloserine, ephedrine, epinephrine, isoniazid, monoamine oxidase inhibitors, nitrates, corticosteroids such as prednisone, reserpine, and synthetic thyroid hormones.

The irritant is included in the composition in an amount at least sufficient to cause significant discomfort when consumed in ways associated with abuse. In immediate-release dosage forms, the irritant is included in a sub-clinical amount which will vary according to a number of factors including the specific irritant selected and the particular application of use. By the inclusion of a sub-clinical amount in such dosage forms, use of such dosage forms at therapeutic dosage levels will expose the patent to an amount of irritant insufficient to cause significant discomfort while use at elevated dosages will result in the abuser being exposed to an amount of irritant sufficient to cause significant discomfort.

In modified-release dosage forms, the irritant is included in each unit dose in an amount sufficient to cause significant discomfort. Significant discomfort is avoided by ingestion of structurally intact modified-release dosage forms due to the sequestration of the irritant included therein. If the structural integrity of the modified-release dosage form and/or the contents thereof has been compromised, the amount of irritant provided in each unit dose is sufficient to cause significant discomfort. In either embodiment, the amount of irritant included in the oral dosage form should be less than an amount which would cause death or serious injury to the average individual. The amount of irritant will vary in accordance with a number of factors including, for example, the particular species of irritant used, the presence of other ingredients, the specific form of the oral dosage formulation, and the particular application in which the composition is intended to be used. It is believed that for most applications, the amount of irritant included in the composition will be from about 0.001 to about 85 wt. %. In preferred form, the amount of irritant included in the composition will be from about 0.001 to about 50 wt. %, and even more preferably from about 0.001 to about 20 wt. %.

The composition of the present invention may include also conventional excipients of the type used in pharmaceutical compositions. For example, the composition may include pharmaceutically acceptable organic or inorganic carriers suitable for oral administration. Examples of such carriers include: sugar spheres, diluents, hydrophilic polymers, film coating polymers, lubricants, glidants (or anti-adherents), plasticizers, binders, disintegrants, surfactants, pH modifiers, preservatives, coloring, flavoring and/or aromatic substances.

Examples of suitable diluents include microcrystalline cellulose; lactose, sucrose, fructose, glucose, dextrose, or other sugars; dibasic calcium phosphate; calcium sulphate; cellulose; ethylcellulose; cellulose derivatives; kaolin; mannitol, lactitol, maltitol, xylitol, sorbitol, or other sugar alcohols; dry starch; dextrin, maltodextrin or other polysaccharides; inositol; or mixtures thereof.

Examples of suitable hydrophilic polymers include hydroxypropylmethyl cellulose; carbomers; polyethylene oxides; hydroxypropyl cellulose; hydroxyethyl cellulose; carboxymethylcellulose; sodium carboxymethylcellulose; carboxyvinylpolymers; polyvinyl alcohols; glucans; scleroglucans; mannans; xanthans; carboxymethylcellulose and its derivatives; methylcellulose; cellulose; crosslinked polyvinylpyrrolidone; carboxymethyl starch; potassium methacrylate-divinylbenzene copolymer; hydroxypropylcyclodextrin; alpha, beta, gamma cyclodextrin or derivatives and other dextran derivatives; natural gums; seaweed extract; plant exudate; agar; agarose; algin; sodium alginate; potassium alginate; carrageenan; kappa-carrageenan; lambda-carrageenan; fucoidan, furcellaran; laminarin; hypnea; eucheuma; gum arabic; gum ghatti; gum karaya; gum tragacanth; guar gum; locust bean gum; quince psyllium; flax seed; okra gum; arabinogalactin; pectin; scleroglucan; dextran; amylose; amylopectin; dextrin; acacia; karaya; guar; a swellable mixture of agar and carboxymethyl cellulose; a swellable composition comprising methyl cellulose mixed with a sparingly cross-linked agar; a blend of sodium alginate; and locust bean gum.

Examples of suitable film-coating polymers include enteric polymer coating materials, such as, for example, cellulose acetate phthalate, cellulose acetate trimaletate, hydroxypropyl methylcellulose phthalate, polyvinyl acetate phthalate, Eudragit® poly acrylic acid and poly acrylate and methacrylate coatings, polyvinyl acetaldiethylamino acetate, hydroxypropyl methylcellulose acetate succinate, cellulose acetate trimellitate, shellac; hydrogels and gel-forming materials, such as, for example, carboxyvinyl polymers, sodium alginate, sodium carmellose, calcium carmellose, sodium carboxymethyl starch, polyvinyl alcohol, hydroxyethyl cellulose, methyl cellulose, gelatin, starch and cellulose-based cross-linked polymers, hydroxypropyl cellulose, hydroxypropyl methylcellulose, polyvinylpyrrolidone, crosslinked starch, microcrystalline cellulose, chitin, cellulose acetate, cellulose proprionate, cellulose acetate propionate, cellulose acetate butyrate, cellulose triacetate, aminoacryl-methacrylate copolymer (Eudragit® RS-PM, Rohm & Haas), pullulan, collagen, casein, agar, gum arabic, sodium carboxymethyl cellulose, carboxymethyl ethyl cellulose, swellable hydrophilic polymers, poly(hydroxyalkyl methacrylate) (m. wt. ˜5 k-5,000 k), polyvinylpyrrolidone (m. wt. ˜10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. ˜30 k-300 k), polysaccharides such as agar, acacia, karaya, tragacanth, algins and guar, polyacrylamides, Polyox® polyethylene oxides (m. wt. ˜100 k-5,000 k), AquaKeep® acrylate polymers, diesters of polyglucan, crosslinked polyvinyl alcohol and poly N-vinyl-2-pyrrolidone, sodium starch glycollate (e.g. Explotab®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, poly(ethylene terphthalate), poly(vinyl isobutyl ether), polyurethane, polyethylene oxides (e.g. Polyox®, Union Carbide), methyl ethyl cellulose, ethylhydroxy ethylcellulose, cellulose acetate, ethylcellulose, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, ethyl acrylate-methyl methacrylate copolymer, sorbitan esters, polydimethyl siloxane, natural gums, lecithins, pectin, alginates, ammonia alginate, sodium, calcium, potassium alginates, propylene glycol alginate, agar, gums: arabic, karaya, locust bean, tragacanth, carrageens, guar, xanthan, scleroglucan and mixtures and blends thereof.

Examples of suitable lubricants include stearic acid, magnesium stearate, talc, calcium stearate, hydrogenated vegetable oils, sodium benzoate, sodium chloride, leucine carbowax, magnesium lauryl sulphate, colloidal silicon dioxide, glyceryl monostearate, waxes, hydrogenated oils, and polyethyleneglycol.

Examples of suitable glidants (or anti-adherents) include colloidal silica, fumed silicon dioxide, silica hydrogel, talc, fumed silica, gypsum, kaolin and glyceryl monostearate. Suitable plasticizers include acetylated monoglycerides, butyl phthalyl butyl glycolate, dibutyl tartrate, diethyl phthalate, dimethyl phthalate, ethyl phthalyl ethyl glycolate, glycerin; propylene glycol, triacetin, citrate, tripropioin, diacetin, dibutyl phthalate, acetyl monoglyceride, polyethylene glycols, castor oil, triethyl citrate, polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, glyceryl monocaprate. Suitable binders include starches, hydroxypropylmethyl cellulose, hydroxypropyl cellulose, ethyl cellulose, polyvinyl pyrrolidone, acacia, guar gum, hydroxyethylcellulose, agar, calcium carrageenan, sodium alginate, gelatin, saccharides (including glucose, sucrose, dextrose and lactose), molasses, extract of Irish moss, panwar gum, ghatti gum, mucilage of isapol husk, carboxymethylcellulose, methylcellulose, veegum, larch arbolactan, polyethylene glycols, waxes and mixtures thereof.

Examples of suitable disintegrants include starches, sodium starch glycollate, crospovidone, croscarmellose, microcrystalline cellulose, low substituted hydroxypropyl cellulose, pectins, potassium methacrylate—divinylbenzene copolymer, polyvinyl alcohol, thylamide, sodium bicarbonate, sodium carbonate, starch derivatives, dextrin, beta cyclodextrin, dextrin derivatives, magnesium oxide, clays, bentonite and mixtures thereof.

Examples of suitable surfactants include nonionic surfactants such as sorbitan sesquioleate, polyoxyethylene sorbitan monooleate, polyoxyethylene monostearate, glycerol monostearate, propylene glycol monolaurate, polyoxyethylene lauryl ether, polyoxyethylene cetyl ether or polyoxyethylene hydrogenated castor oil, and ionic surfactants such as sodium dodecyl sulfate or benzalkonium chloride.

Examples of suitable pH modifiers include organic acids such as citric acid, fumaric acid, tartaric acid, succinic acid, ascorbic acid, acetic acid, malic acid, glutaric acid and adipic acid; salts of these acids; salts of inorganic acids and magnesium hydroxide.

Another aspect of the present invention is an oral dosage form comprising an opiate and an irritant. The dosage form may be provided in any form that is suitable for the oral administration of an opiate composition. For example, the dosage form may be a tablet, capsule, sprinkle or multiparticulate formulation (that is, granules, spheroids, beads, pellets or the like). The dosage form of the present invention may be provided also as gelatin capsules.

In a preferred embodiment, the dosage form is a tablet. In such embodiment, the tablet may be uncoated or it may be coated by known techniques for a variety of purposes including, for example, employment of a modified release feature, protection of the composition, or improvement of the aesthetics of the tablet.

In a further preferred embodiment, the dosage form is a multiparticulate dosage form. In such embodiment, the individual particles (i.e., granules, spheroids, beads, pellets or the like) can be uncoated or they can be coated by known techniques or there can be a combination of coated and uncoated particles or a combination of differently coated particles. In such embodiment, the irritant and the opiate can each be provided in different beads or they can be present in the same bead. For example, there can be one or more populations of particles containing the opiate and not the irritant, and one or more populations of particles containing the irritant and not the opiate. The different populations can then be mixed in the desired ratios before being filled into a final dosage form such as a capsule or sprinkle. Similarly, there may be one or more populations of particles that contain both the opiate and the irritant. Such analgesic/irritant populations of particles can be mixed together prior to being filled into a final dosage form such as a capsule or sprinkle or can be mixed with one or more populations that contain the opiate but not the irritant and/or the irritant but not the opiate prior to being filled into a final dosage form such as a capsule or a sprinkle.

It is preferred that the dosage form be formulated to have a modified-release property. The term “modified release” is defined for purposes of the present invention as the release of the opiate-based analgesic at a rate such that the plasma concentration of the analgesic within the person to whom the analgesic has been administered is maintained within an acceptable therapeutic range, that is, above a minimum therapeutically effective analgesic concentration but below toxic levels, over the period of time in which the opiate is released. The modified-release property of the oral dosage form of the present invention may be achieved in any number of ways that are available in the art. For example, there can be used a modified-release carrier which is incorporated into the matrix of the composition, or a modified-release coating applied to surface of the dosage form. In those embodiments which employ a modified-release coating, the coating material is selected to achieve the desired in-vitro release rate and should be capable preferably of forming a strong, continuous film that is smooth and elegant, and able to support colorants and other coating additives. In addition, the coating material should preferably be non-toxic, inert, and tack-free.

In a preferred embodiment, the modified-release coatings permit either pH-dependent or pH-independent release of the analgesic, for example, when exposed to the gastrointestinal liquids. A pH-dependent coating serves to release the opiate in desired locations of the GI tract, for example, the stomach or small intestine, such that there is provided an absorption profile which is capable of providing in the user a sustained release of opiate, for example, at least about 1 hour up to about 30 hours. When a pH-independent coating is desired, the coating is designed to achieve optimal release regardless of pH variations along the GI tract. It is also possible to formulate compositions which release a portion of the unit dose in one desired location of the GI tract, for example, the stomach, and release the remainder of the unit dose in another location of the GI tract, for example, the small intestine.

An oral dosage form according to the present invention that utilizes pH-dependent coatings may also impart a repeat-action effect in which a portion of the opiate overlies an enteric coating and is released in the stomach and the remaining portion of the opiate is protected by the enteric coating and is released further along the GI tract. Coatings which are pH-dependent may be formed, for example, from shellac, cellulose acetate phthalate, polyvinyl acetate phthalate, hydroxypropylmethylcellulose phthalate, and methacrylic acid ester copolymers, zein, cellulose acetate trimaletate, poly acrylic acid and poly acrylate, polyvinyl acetaldiethylamino acetate, hydroxypropyl methylcellulose acetate succinate and cellulose acetate trimellitate.

In accordance with the present invention, ingestion of immediate-release oral dosage forms of the present invention at therapeutic dosage levels and modified-release dosage forms in a manner that does not defeat the modified-release properties thereof will not result in significant discomfort to the patient. By contrast, when the dosage level of immediate-release forms is exceeded or the structural integrity of modified-release dosage forms is destroyed, such as by chewing, crushing or dissolving, and the composition is consumed orally, nasally, or by injection, the irritant is exposed to the body of the abuser in an amount sufficient to cause significant discomfort, either locally and/or systemically. It is the discomfort caused by the irritant that serves to deter abuse of the oral dosage form of the composition of the present invention.

We claim:

1. A pharmaceutical composition comprising a therapeutically effective amount of an opiate and an irritant.

2. The composition of claim 1 wherein the irritant is a local irritant.

3. The composition of claim 2 wherein the irritant is a capsaicinoid.

4. The composition of claim 3 wherein the capsaicinoid is capsaicin.

5. The composition of claim 1 wherein the irritant is a systemic irritant.

6. The composition of claim 1 wherein the irritant is capable of causing significant discomfort to the gastrointestinal system of a human.

7. The composition of claim 1 wherein the irritant is an emetic.

8. The composition of claim 1 wherein the systemic irritant is capable of causing significant discomfort to the immune system of a human.

9. The composition of claim 1 wherein the irritant is a histamine.

10. The composition of claim 1 wherein the opiate comprises from about 0.1 to about 40 percent by weight and the irritant comprises from about 0.001 to about 85 percent by weight of the composition.

11. The composition of claim 1 wherein the opiate is selected from the group consisting of codeine, hydrocodone, hydromorphone, levorphanol, meperidine, morphine, oxycodone, and pharmaceutically acceptable salts thereof and the irritant is a local irritant.

12. The composition of claim 1 wherein the opiate is selected from the group consisting of codeine, hydrocodone, hydromorphone, levorphanol, meperidine, morphine, oxycodone, and pharmaceutically acceptable salts thereof and the irritant is a systemic irritant.

13. The composition of claim 1 wherein the irritant is sequestered.

14. The composition of claim 13 wherein the irritant is coated by a material that is substantially indigestible.

15. The composition of claim 13 wherein the irritant is chemically bound to a material that renders the irritant biologically unavailable.

16. An oral dosage form comprising the composition of claim 1.

17. The oral dosage form of claim 16 wherein the irritant is present in a sub-clinical amount.

18. The oral dosage form of claim 17 wherein the opiate is provided in immediate-release form.

19. The oral dosage form of claim 16 wherein the opiate is provided in modified-release form and the irritant is sequestered.

20. The oral dosage form of claim 16 wherein the dosage is in tablet form.

21. The oral dosage form of claim 16 wherein the dosage is in a multiparticulate form.

22. A method of deterring abuse of an opiate comprising combining a therapeutically effective amount of an opiate and an irritant into an oral dosage formulation.

23. The method of claim 22 wherein the irritant is provided in a sub-clinical amount.

24. The method of claim 23 wherein the opiate is provided in immediate-release form.

25. The method of claim 22 wherein the opiate is provided in modified-release form and the irritant is sequestered.

26. A method of treating pain comprising orally administering to an individual a therapeutically effective amount of an opiate and an irritant.

27. The method of claim 26 wherein the irritant is provided in a sub-clinical amount.

28. The method of claim 27 wherein the opiate is provided in immediate-release form.

29. The method of claim 26 wherein the opiate is provided in modified-release form and the irritant is sequestered.
